(19) United States
(12) Patent Application Publication
(10) Pub. No.: US 2025/0073683 A1
(43) Pub. Date: Mar. 6, 2025

(54) Metal Oxide Compounds with Enhanced Reactivity

(71) Applicant: NANOIONIX, LLC, Farmington, CT (US)

(72) Inventors: Lawrence H. Dubois, Danbury, CT (US); Dana Alexa Totir, Sandy Hook, CT (US)

(21) Appl. No.: 18/822,019

(22) Filed: Aug. 30, 2024

Related U.S. Application Data
(60) Provisional application No. 63/535,538, filed on Aug. 30, 2023.

(51) Int. Cl. B01J 23/34; A01N 59/16; A01P 1/00; A61L 2/232; B01J 23/00; B01J 23/889; B01J 35/50; C02F 1/50; C02F 1/72; C02F 101/34; C02F 101/38

(52) U.S. Cl. CPC B01J 23/34; A01N 59/16; A01P 1/00; A61L 2/232; B01J 23/002; B01J 23/8892; B01J 35/505; C02F 1/50; C02F 1/725; C02F 2101/34; C02F 2101/38; C02F 2303/04; C02F 2303/20

(57) ABSTRACT

Metal oxide ceramic compounds are described that have a high capacity for oxidation of chemical or biological entities through the generation of reactive oxygen species spontaneously without the need for light, heat, electricity, or additional chemical constituents. These materials have at least one metal ion which displays multiple valence states. Applications include the decomposition of toxic chemicals, the inactivation of pathogens, and the prevention of biofouling.

### CROSS-REFERENCE TO RELATED APPLICATION

The benefit under 35 USC § 119 of U.S. Provisional Patent Application 63/535,538 filed Aug. 30, 2023, in the names of Lawrence H. Dubois and Dana Totir for “Enhanced Generation of Reactive Oxygen Species on Mixed Metal Oxide Ceramics” is hereby claimed, and the disclosure thereof is hereby incorporated herein by reference, in its entirety, for all purposes.

### FIELD OF THE DISCLOSURE

The present disclosure relates generally to metal oxide ceramic compounds, compositions and materials containing same, and methods of making and using same. More specifically, the disclosure relates in various aspects to metal oxide ceramic compounds that generate reactive oxygen species spontaneously without the need for light, heat, electricity, or additional chemical constituents. Such metal oxide ceramic compounds include at least one metal ion that displays multiple valence states. The metal oxide compounds of the disclosure may be utilized in applications such as the decomposition of toxic chemicals, the inactivation of pathogens, and the prevention of biofouling.

### BACKGROUND

Reactive oxygen species (ROS) such as hydrogen peroxide (H₂O₂), singlet oxygen (¹O₂), superoxide ions (O₂·⁻), and hydroxyl radicals (HO·) are known to decompose toxic chemicals, to inactivate pathogens including bacteria, viruses, and fungi, and to minimize biofouling of underwater surfaces. While ROS are produced biologically in the human body as a natural defense again foreign matter, they can also be generated by inorganic materials. In the presence of water, oxygen, and an organic substituent, ROS are produced spontaneously on the surfaces of metal oxides (for example by perovskites such as lanthanum strontium manganese oxide; U.S. Pat. No. 11,572,285), in the presence of water and ultraviolet radiation (for example by titanium dioxide nanoparticles; Antimicrobial Effect of Titanium Dioxide Nanoparticles (2019) IntechOpen), electrochemically in solution (for example by zinc gallium oxide; Nature Comm. (2023) 14:1890), and by galvanic corrosion in air or water (for example, silver-ruthenium: J. Hazard. Mater. 440 (2022) 129730).

The effectiveness of toxic chemical decomposition, pathogen inactivation, or biofouling prevention resulting from ROS is determined by the type and number of ROS that are generated. This in turn is determined by the chemistry—stoichiometry, doping, particle size/particle shape, number and type of defects, etc.—of the active material (and the intensity and wavelength of light, potential and current, and/or additional chemical constituents, if required for ROS production by such active material).

Calcium manganese oxides (CaₓMn_{y}O_{z}) are a family of mixed valence metal oxide (Ca⁺²; O⁻²; Mn⁺²/Mn⁺³/Mn⁺⁴) ceramic materials with short-range order, a layered perovskite or spinel structure, and low surface energy. Oxygen is bonded in several multi-coordinate sites yielding a distortion in the crystal lattice with Ca—O and Mn—O bond lengths varying by up to 0.03 nm. This leads to relatively weak water binding, rapid surface diffusion to active sites, and facile electron transfer between manganese in different oxidation states (PNAS 110 (2013) 8801). Because of this, calcium manganese oxides are considered good model systems for photocatalytic water oxidation and oxygen generation in photosystem II.

Due to the stable perovskite lattice, CaₓMn_{y}O_{z} may be doped with a variety of ions to control its electrical, thermal, catalytic, and photocatalytic properties. For example, calcium manganese titanium oxide is a black pigment with high infrared reflectivity (U.S. Pat. No. 8,906,272). The synthesis of CaₓMn_{y}O_{z} and doped-CaₓMn_{y}O_{z} is straightforward using solid-state, solution, and hydrothermal processes, and the chemical and catalytic properties of CaₓMn_{y}O_{z} have been studied for over 40 years, but in all such studies and synthetic efforts, energy in the form of light, heat, electricity, and/or chemical oxidants is required for the CaₓMn_{y}O_{z} or doped-CaxMnyOz to be catalytically active.

It would correspondingly be highly advantageous to provide CaₓMn_{y}O_{z}-based compositions and other metal oxide compositions that are effective to generate reactive oxygen species and provide oxidizing properties in the absence of light, heat, electricity, and/or chemical oxidants that heretofore have been required to mediate oxidative and catalytic activity.

### SUMMARY

The present disclosure relates to metal oxide ceramic compounds, compositions and materials containing same, and methods of making and using same.

In one aspect, the disclosure relates to a material or blend of materials with an average stoichiometry CaₓMn_{y}O_{z} wherein x≥0.1, y≥0.1, and z≥1, which generates spontaneously reactive oxygen species.

Another aspect of the disclosure relates to a method of decomposing a chemical, inactivating a pathogen, or preventing biofouling at a biofouling-susceptible locus, comprising contacting the chemical, pathogen, or biofouling-susceptible locus with the material or blend of materials described in the immediately preceding paragraph.

A further aspect of the disclosure relates to a material or blend of materials with an average stoichiometry AeₓTm_{y}O^{z} wherein Ae is one or more alkaline earth elements, Tm is one or more transition metals or lanthanides with at least one having multiple valences, and x≥0.1, y≥0.1, and z≥1, which generates spontaneously reactive oxygen species.

Another aspect of the disclosure relates to a method of decomposing a chemical, inactivating a pathogen, or preventing biofouling at a biofouling-susceptible locus, comprising contacting the chemical, pathogen, or biofouling-susceptible locus with the material or blend of materials described in the immediately preceding paragraph.

Other aspects, features, and embodiments of the disclosure will be more fully apparent from the ensuing description and appended claims.

### BRIEF DESCRIPTION OF THE DRAWINGS

FIG. 1 in (a) shows a representative powder X-ray diffraction pattern confirming the perovskite structure of a sub-stoichiometric CaMnO_{3-δ} prepared by a solution-based synthesis procedure and in (b) shows a scanning electron micrograph of such sub-stoichiometric CaMnO_{3-δ} in which the particle shape and size of such sub-stoichiometric CaMnO_{3-δ} is observable.

FIG. 2 shows in (a) the spontaneous generation of H₂O₂ on CaMnO_{3-δ} as detected by UV-Vis spectroscopy using leuco crystal violet as a selective trapping agent, at room temperature (˜25° C.), and in (b) shows the spontaneous generation of ¹O₂ from the same material detected by fluorescence spectroscopy using singlet oxygen sensor green (SOSG) as a selective trapping agent (excitation max 480 nm, emission max 528 nm) at room temperature (˜25° C.).

FIG. 3 in (a) shows a graph of absorbance as a function of wavelength, in nanometers, for decomposition of acetaminophen in the presence of CaMnO_{3-δ} at room temperature, as detected by UV-Vis spectroscopy, and in (b) shows a graph of absorbance as a function of wavelength, in nanometers, for decomposition of tryptophan in the presence of CaMnO_{3-δ} at room temperature, as detected by UV-Vis spectroscopy.

FIG. 4 in (a) shows a powder x-ray diffraction pattern of CaMnO_{3-δ} synthesized by a sol-gel procedure, in (b) shows a powder X-ray diffraction pattern of CaMnO_{3-δ} synthesized by a solid-state procedure, in (c) shows a scanning electron micrograph of CaMnO_{3-δ} synthesized by the sol-gel procedure, and in (d) shows a scanning electron micrograph of CaMnO_{3-δ} synthesized by the solid-state procedure.

FIG. 5 in (a) shows powder x-ray diffraction patterns as a function of Ca:Mn ratio in CaMnO_{3-δ}, ranging from pure perovskite to a mix of pure perovskite and Ruddlesden-Popper layered perovskite, and in (b) depicts a bar graph of H₂O₂ concentration, in ppm, for the calcium manganese oxide CaMnO_{3-δ} compounds identified in such graph, showing the variation in spontaneous generation of hydrogen peroxide as a function of the Ca:Mn ratio, as detected using UV-Vis spectroscopy with leuco crystal violet as a selective trapping agent, and demonstrating that hydrogen peroxide production was maximized with a mixture of pure perovskite and Ruddlesden-Popper layered perovskite.

FIG. 6 in (a) thereof shows the powder X-ray diffraction patterns of CaTi_{0.1}Mn0.9O₃ (top) and Ca₂Ti0.1Mn_{0.9}O₄ (bottom) synthesized by the solid-state procedure, and scanning electron micrographs are shown in (b) of FIG. 6 for such CaTi_{0.1}Mn0.9O₃ and Ca₂Ti0.1Mn_{0.9}O₄ compounds.

FIG. 7 shows a representative powder X-ray diffraction pattern of CuMnO₃ synthesized by a solid-state process confirming the perovskite structure.

FIG. 8 in (a) thereof shows a scanning electron micrograph of CaMnO_{3-δ} blended with a polyurethane/polyurea binder and coated at 10% by weight on an aluminum foil disc, and in (b) shows an energy dispersive X-ray analysis of both calcium (red) and manganese (purple) evidencing a uniform distribution of ceramic particles across the coating.

FIG. 9 top panel shows a powder X-ray diffraction pattern of ˜10 micron diameter rutile TiO₂ particles coated with CaMnOs by a sol-gel process. The middle and lower panels are the diffraction patterns of pure rutile TiO₂ and CaMnO₃, respectively.

### DETAILED DESCRIPTION

The present disclosure provides metal oxide ceramic compounds that are effective to generate reactive oxygen species and provide oxidizing properties in the absence of light, heat, electricity, and/or chemical oxidants, although light, heat, electricity, and/or chemical oxidants may additionally be employed with such metal oxide ceramic compounds to augment or enhance their ROS-generating and oxidizing properties.

The present disclosure is based on the discovery that with the proper stoichiometry and structure, spontaneous generation of reactive oxygen species may be observed when CaₓMn_{y}O_{z}, doped CaₓMn_{y}O_{z}, and related compounds such as CaₓFe_{y}O_{z}, CuₓMn_{y}O_{z}, or CeₓMn_{y}O_{z} wherein at least one metal ion can exist in multiple oxidation states, are exposed to oxygen (air), water (humidity), and an organic moiety including toxic chemicals and biological species (bacteria, virus, fungi, algae) at room temperature (e.g., ˜25° C.), or other ambient temperature range such as 10° C. to 40° C.—no light, heat, electricity, or chemical additives are required, although their use and/or addition is not precluded.

The present disclosure further reflects the finding that while CaₓMn_{y}O_{z} has only one metal ion (manganese) that can be present in a mixed valence state, adding a second metal ion with multiple valence states and/or replacing calcium with one or metals such as vanadium, chromium, iron, cerium, cobalt, nickel, and/or copper can introduce strong oxidizing properties while maintaining the capacity to spontaneously generate ROS.

Applications of the metal oxide compounds of the present disclosure include the decomposition of toxic chemicals, the inactivation of pathogens, and the prevention of biofouling.

As used herein in reference to a material or blend of materials, “generates spontaneously reactive oxygen species” means that the material or blend of materials spontaneously produces reactive oxygen species in the presence of (i) oxygen or oxygen-containing gas, e.g., air, (ii) water, e.g., humidity, and (iii) an organic moiety selected from among organic chemicals, e.g., acetaminophen or tryptophan, and biological species (bacteria, virus, fungi, algae), at temperature in a range of 10° C. to 40° C., and in the absence of added or inputted light, heat, electricity, or chemical additives.

As used herein, “material capable of a Fenton reaction” means a material that reacts with hydrogen peroxide to form hydroxyl radicals that are effective to decompose organic or other materials, e.g., chemicals, and pathogenic species.

In one aspect, the disclosure relates to a material or blend of materials with an average stoichiometry CaₓMn_{y}O_{z} wherein x≥0.1, y≥0.1, and z≥1, which generates spontaneously reactive oxygen species.

In various embodiments, such material or blend of materials may be additionally blended with a material capable of a Fenton reaction.

In other embodiments, such material or blend of materials may have added thereto one or more alkali metal, alkaline earth, transition metal, lanthanide, and/or main group element(s). Alkali metal elements useful for such purpose include lithium, sodium, potassium, rubidium, and cesium. Alkaline earth elements useful for such purpose include magnesium, strontium, and barium. Transition elements that may be employed include scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, and gold. Lanthanide elements for such purpose include cerium, praseodymium, neodymium, promethium, samarium, europium, gadolinium, terbium, dysprosium, holmium, erbium, thulium, ytterbium, and lutetium. Main group elements that may be useful for such purpose include elements of Groups 1, 2, and 13 to 18 of the Periodic Table.

In various embodiments, the above-described material or blend of materials may be constituted, wherein at least some of the oxygen is replaced by one or more halogen and/or chalcogenide atoms. Halogen atoms suitable for such replacement include chlorine, fluorine, bromine, and iodine. Chalcogenide atoms that may be used for such replacement include sulfur, selenium, and tellurium.

In other embodiments, the material or blend of materials of the disclosure may comprise a CaₓMn_{y}O_{z} perovskite lattice substituted with one or more A-site cation(s) selected from the group consisting of strontium, barium, sodium, potassium, lanthanum, cerium, and bismuth.

In various embodiments, the material or blend of materials of the disclosure may comprise a CaₓMn_{y}O_{z} perovskite lattice substituted with one or more B-site cation(s) selected from the group consisting of titanium, vanadium, iron, nickel, copper, zinc, cerium, aluminum, and antimony.

In various embodiments, the material or blend of materials of the disclosure may comprise a CaₓMn_{y}O_{z} perovskite lattice substituted with one or more anion(s) selected from the group consisting of sulfur, fluorine, and chlorine.

In various embodiments, the material or blend of materials may variously include any one or more of the A-site substitutions and/or B-site substitutions and/or anion substitutions described in the preceding three paragraphs, with respect to substitution(s) selected from the group consisting of the specified A-site cation(s), B-site cation(s), and anion(s).

The material or blend of materials of the disclosure may be coated on the surface of an object, or coated on the surface of a particle, or mixed with a binder and coated on a surface.

In a further aspect, the disclosure relates to a method of decomposing a chemical, inactivating a pathogen, or preventing biofouling at a biofouling-susceptible locus, comprising contacting the chemical, pathogen, or biofouling-susceptible locus with the material or blend of materials of the present disclosure, as variously described above.

Another aspect of the disclosure relates to a material or blend of materials with an average stoichiometry AeₓTm_{y}O_{z} wherein Ae is one or more alkaline earth elements, Tm is one or more transition metals or lanthanides with at least one having multiple valences, and x≥0.1, y≥0.1, and z≥1, which generates spontaneously reactive oxygen species. The respective alkaline earth elements, transition metals, and lanthanides may include any the specific types of alkaline earth elements, transition metals, and lanthanides mentioned in the preceding description.

The material or blend of materials with an average stoichiometry AeₓTm_{y}O_{z} may be blended with a material capable of a Fenton reaction.

Such material or blend of materials with an average stoichiometry AeₓTm_{y}O_{z} may be coated on the surface of an object, or on the surface of a particle, or may be mixed with a binder and coated on a surface, in various respective embodiments.

In particular embodiments, the material or blend of materials with an average stoichiometry AeₓTm_{y}O_{z} may have added thereto one or more alkali metal, alkaline earth, transition metal, lanthanide, and/or main group element(s). Specific alkali metal, alkaline earth, transition metal, lanthanide, and/or main group element(s) include those previously described herein.

In various embodiments, the material or blend of materials with an average stoichiometry AeₓTm_{y}O_{z} may be constituted, wherein at least some of the oxygen is replaced by one or more halogen and/or chalcogenide atoms. Suitable halogen and/or chalcogenide species for such purpose include the halogens and chalcogenides previously discussed herein.

The material or blend of materials with an average stoichiometry AeₓTm_{y}O_{z} may be employed in a method of decomposing a chemical, inactivating a pathogen, or preventing biofouling at a biofouling-susceptible locus, in which the chemical, pathogen, or biofouling-susceptible locus is contacted with such material or blend of materials.

The features and advantages of the present disclosure are more fully shown by the following non-limiting Examples, as illustrative of specific implementations of the disclosure in corresponding embodiments thereof.

### EXAMPLE 1

Sub-stoichiometric CaMnO_{3-δ} was synthesized by a co-precipitation process. Ca(NO₃)₂·4H₂O and Mn(NO₃)₂·4H₂O were dissolved in distilled water in a 1:1 ratio and stirred for 20 minutes. A 0.5M solution of (NH₄)₂CO₃ was added in 3:1 excess and the resulting precipitate was filtered and double washed with warm distilled water, then dried for 18 hours at 80° C., ground in a mortar and pestle, and calcined from 2 10 12 hours at temperatures between 400 and 900° C.

Mixed manganese valence states are critical for ROS generation. Stoichiometric CaMnO₃ has an average Mn oxidation state of +4 (which does not imply that all manganese ions are in that state). Under the above-described synthesis conditions, the resulting calcium manganese oxide is deficient in oxygen resulting in an average Mn oxidation state of ˜+3.8.

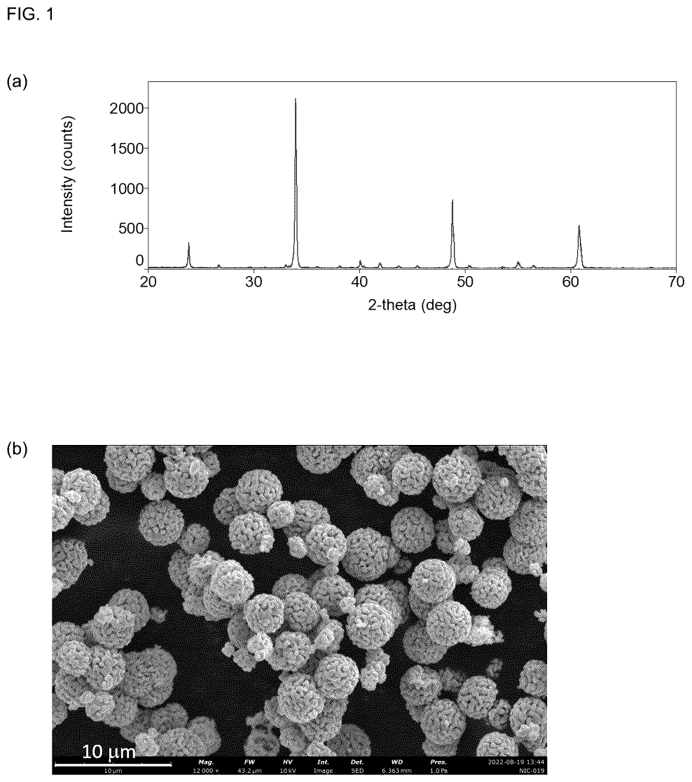

FIG. 1 in (a) shows a representative powder X-ray diffraction pattern confirming the perovskite structure and in (b) shows a scanning electron micrograph of the sub-stoichiometric CaMnO_{3-δ} from the foregoing solution-based synthesis procedure, in which the particle shape and size of such sub-stoichiometric CaMnO_{3-δ} is observable.

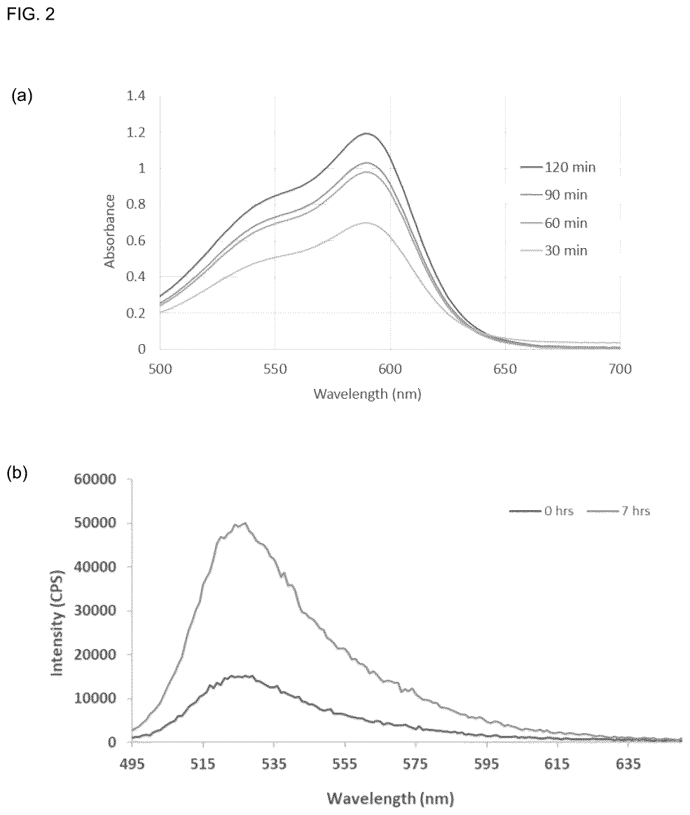

The sub-stoichiometric CaMnO_{3-δ} was tested to determine its capability for spontaneous generation of H₂O₂ using UV-Vis spectroscopy with leuco crystal violet as a selective trapping agent, at room temperature (˜25° C.), with the result shown in graph (a) in FIG. 2, of absorbance as a function of wavelength, in nanometers, at time intervals of 30 minutes, 60 minutes, 90 minutes, and 120 minutes.

The sub-stoichiometric CaMnO_{3-δ} was also tested to determine its capability for spontaneous generation of ¹O₂ at room temperature (˜25° C.), by fluorescence spectroscopy using singlet oxygen sensor green (SOSG) as a selective trapping agent (excitation max 480 nm, emission max 528 nm). The results are shown in graph (b) of FIG. 2, in which fluorescence intensity (counts per second, CPS) is plotted as a function of wavelength, in nanometers, at time zero (0 hours) and at 7 hours.

The data shown in FIG. 2 in (a) thereof thus demonstrate the spontaneous generation of H₂O₂ on the sub-stoichiometric CaMnO_{3-δ}. It is noted that a Fenton catalyst (a catalyst that converts H₂O₂ to 2 OH·, such as for example Fe₂O₃) may be blended with the sub-stoichiometric CaMnO_{3-δ}, to convert some or all of the generated hydrogen peroxide to the more reactive OH·.

The data shown in FIG. 2 in (b) thereof demonstrate the spontaneous generation of ¹O₂ from the sub-stoichiometric CaMnO_{3-δ}. ¹O₂ may be a primary product of the reaction of the sub-stoichiometric CaMnO_{3-δ} with oxygen, water, and organic species or may be a decomposition product of the hydrogen peroxide that is generated. It is noted that CaMnO_{3-δ} readily reacts with H₂O₂ at room temperature to generate oxygen bubbles.

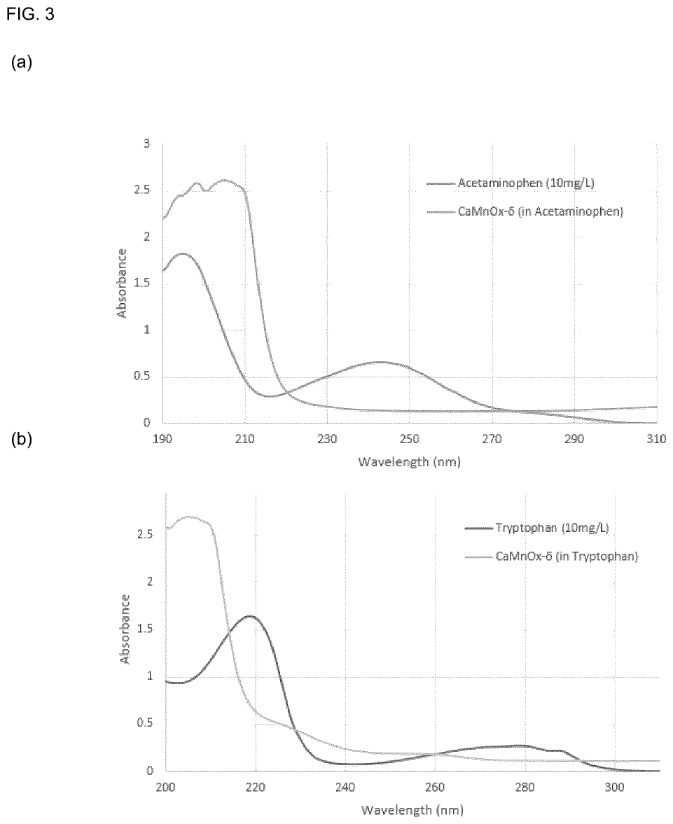

Testing was also conducted using the sub-stoichiometric CaMnO_{3-δ} to determine its capability to effect decomposition of acetaminophen, a representative pharmaceutical residue found in wastewater, at room temperature (˜25° C.). 2.5 g of CaMnO_{3-δ} was added to 50 ml of a 10 mg/L solution of acetaminophen. UV-Vis spectroscopy spectra were recorded at time zero (0 hours) and after stirring for 4 hours, evidencing decomposition of acetaminophen, as shown in the spectral data graph (a) in FIG. 3, of absorbance as a function of wavelength in nanometers, for the acetaminophen solution (10 mg/liter) without the sub-stoichiometric CaMnO_{3-δ}, and for the acetaminophen solution containing the sub-stoichiometric CaMnO_{3-δ}.

The sub-stoichiometric CaMnO_{3-δ} was also tested to determine its capability for decomposing tryptophan, a prototypical amino acid, at room temperature (˜25° C.). 2.5 grams of CaMnO_{3-δ} was added to 50 ml of a 10 mg/L solution of tryptophan. UV-Vis spectroscopy spectra were recorded at time zero (0 hours) and after stirring for 4 hours, evidencing decomposition of tryptophan, as shown in the spectral data graph (b) in FIG. 3, of absorbance as a function of wavelength in nanometers, for the tryptophan solution (10 mg/liter) without the sub-stoichiometric CaMnO_{3-δ}, and for the tryptophan solution containing the sub-stoichiometric CaMnO_{3-δ}.

The results shown in FIG. 3 confirmed the capability of the sub-stoichiometric CaMnO_{3-δ} to decompose acetaminophen and to decompose tryptophan.

### EXAMPLE 2

Particle size, particle size distribution, and particle shape may be controlled by the synthesis procedure. For sol-gel synthesis, stoichiometric amounts of Ca (NO₃)₂·4H₂O and Mn (NO₃)₂·4H₂O were dissolved in distilled water, and citric acid was added as a ligand, in 2:1 mole ratio to the metal ions. The solution was heated in an oil bath at 90° C. for 4 hours to evaporate the solvent and promote polymerization until a spongy orange solid was formed. The resulting mass was dried at 180° C. for 20 hours, ground in a mortar and pestle, and then calcined at 600° C. for 4 hours, yielding the sub-stoichiometric CaMnO_{3-δ} product. For the solid-state synthesis, stoichiometric amounts of CaCO₃ and MnCO₃ were mixed for 1 hour in a mortar and pestle and then calcined at 1200° C. for 12 hours, yielding the sub-stoichiometric CaMnO_{3-δ} product.

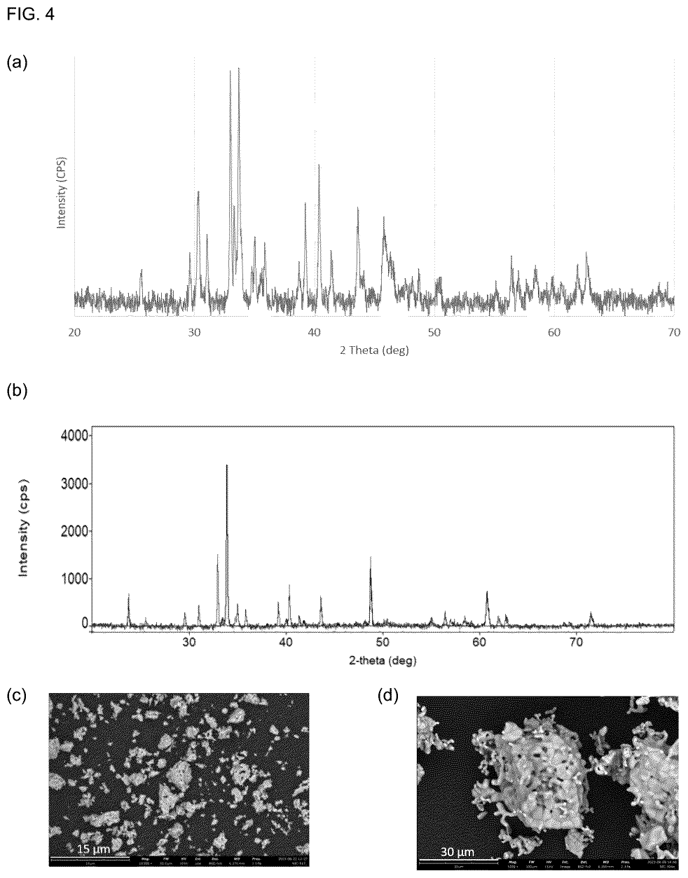

FIG. 4 shows powder X-ray diffraction patterns of (a) the sub-stoichiometric CaMnO_{3-δ} product synthesized by the sol gel synthesis procedure, and (b) the sub-stoichiometric CaMnO_{3-δ} product synthesized by the solid-state synthesis procedure, as well as scanning electron micrographs of (c) the sub-stoichiometric CaMnO_{3-δ} product synthesized by the sol gel synthesis procedure, and (d) the sub-stoichiometric CaMnO_{3-δ} product synthesized by the solid-state synthesis procedure. The scanning electron micrographs show that the particle size for the sub-stoichiometric CaMnO_{3-δ} product synthesized by the solid-state procedure is larger than that of the sub-stoichiometric CaMnO_{3-δ} product synthesized by the sol-gel process.

### EXAMPLE 3

Since the oxidation state of calcium and oxygen are fixed at +2 and −2, respectively, if x, y, and z are known in CaₓMn_{y}O_{z}, then the average oxidation state of manganese is equal to (2*z−2*x)/y. The ratio of x to y may be varied by adjusting the Ca to Mn ratio in the starting materials, as shown in Table 1 below.

The oxygen stoichiometry (z) may be controlled by adding or removing oxygen during the calcining process or by post treatment (for example, by controlled heating (Inorg. Chem. 53 (2014) 9106)). This too changes the average oxidation state of manganese, as shown in Table 1 below.

TABLE 1
Control of theoretical average manganese oxidation state
in CaMnO₃ via stoichiometry. Left table: impact of
changes in calcium:manganese atomic ratio. Right table:
impact of changes in oxygen stoichiometry.

| CaₓMnO₃ | | CaMnO_{z} | |
|---|---|---|---|
| Ca Stoichiometry | Theoretical Mn | O Stoichiometry | Theoretical Mn |
| (x) | Oxidation State | (z) | Oxidation State |
| 0.9 | 4.2 | 2.4 | 2.8 |
| 1.0 | 4.0 | 2.6 | 3.2 |
| 1.1 | 3.8 | 2.8 | 3.6 |
| 1.2 | 3.6 | 3.0 | 4.0 |
| 1.3 | 3.4 | 3.2 | 4.4 |

CaₓMn_{y}O_{z} with ratios of Ca:Mn of 1:1.4, 1:1.2, 1:1, 1:0.8, 1:0.6, and 1:0.5 were synthesized by a hybrid solution-solid state procedure. Stoichiometric amounts of Ca(C₂H₃O₂)₂·H₂O and Mn(C₂H₃O₂)₂·4H₂O were dissolved in distilled water and stirred for 20 minutes, then the mixed solution was evaporated in a drying oven at 80° C. overnight. The resulting mix of salts was ground in a mortar and pestle and heated at 400° C. for 2 hours to decompose the acetate ligands, followed by calcination at 1000° C. for 2 hours.

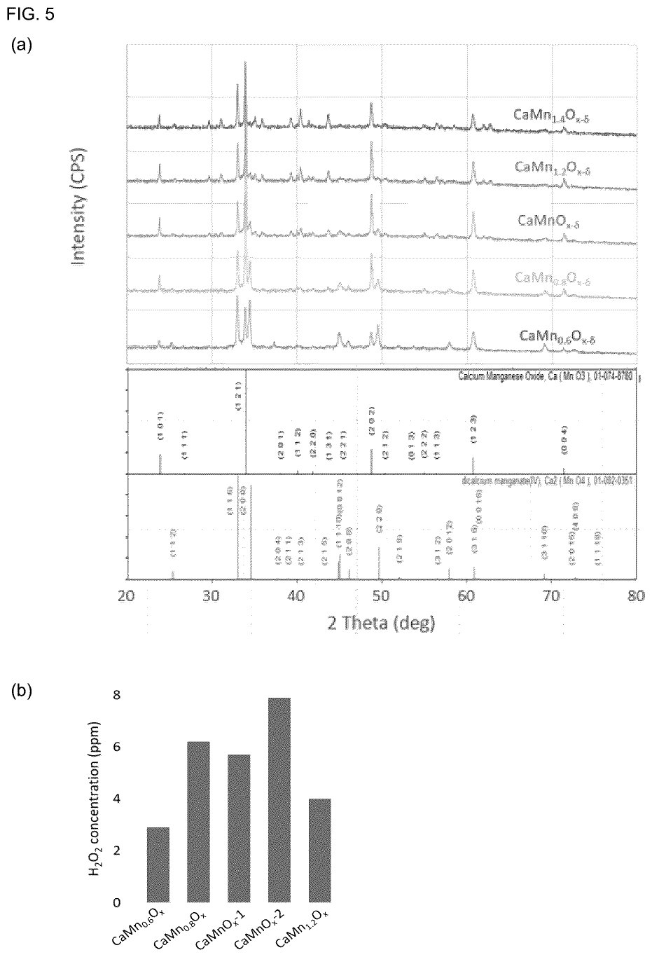

The respective CaₓMn_{y}O_{z} products were assessed by powder X-ray diffraction, with the resulting powder X-ray diffraction patterns (intensity (CPS) as a function of 2 theta (degrees)) shown in (a) of FIG. 5. The powder x-ray diffraction patterns shown in (a) of FIG. 5 varied as a function of the Ca:Mn ratio from pure perovskite to a mix of pure perovskite and Ruddlesden-Popper layered perovskite. The Ruddlesden-Popper phase consists of two-dimensional perovskite-like layers interleaved with calcium cations and shows high structural flexibility in accommodating oxygen vacancies/oxygen non-stoichiometry. X-ray diffraction patterns of the two individual materials, calcium manganese oxide and dicalcium manganate, are shown at the bottom of (a) in FIG. 5 for reference.

FIG. 5 in (b) thereof is a bar graph of H₂O₂ concentration, in ppm, for the calcium manganese oxide compounds identified in such graph, showing the variation in spontaneous generation of hydrogen peroxide as a function of the Ca:Mn ratio, for H₂O₂ production detected using UV-Vis spectroscopy with leuco crystal violet as a selective trapping agent. Hydrogen peroxide production was maximized with a mixture of pure perovskite and Ruddlesden-Popper layered perovskite. As noted in Example 1, a Fenton catalyst such as Fe₂O₃ may be blended with CaₓMn_{y}O_{z} to convert some or all the hydrogen peroxide to the more reactive OH·.

### EXAMPLE 4

While mixed valence states in manganese are central to the spontaneous generation of ROS, other factors are also important. This includes oxygen vacancies, which both alter the surface chemistry and control the diffusion rate of oxygen in the lattice, and electrical conductivity which allows electrons and holes to rapidly move both within the crystal lattice as well as on the surface. Calcium (or other “A” site constituents) also play a role as pure manganese oxides with a mixed valence state (e.g., Mn₃O₄ with Mn⁺²/Mn⁺³) do not generate comparable quantities of ROS.

Thus, the properties of CaₓMn_{y}O_{z} may be modified by substitution at the calcium, manganese, and/or oxygen sites with (i) ions of the same valence, but different ionic radius (larger or smaller, which distort the perovskite lattice), (ii) ions of similar ionic radius, but different valence (this too can distort the lattice due to changes in the metal-oxygen bond length), or (iii) both (i) and (ii).

Table 2 below shows the ionic radius of various ions that may be substituted into the CaₓMn_{y}O_{z} lattice. Metal ions such as vanadium, nickel, iron, copper, cerium, etc. may also be substituted into the A site to form compounds such as vanadium manganese oxide, nickel manganese oxide, iron manganese oxide, copper manganese oxide, cerium manganese oxide, etc., of varying stoichiometries.

TABLE 2
Ionic radius of representative ions that can substitute
into the CaₓMn_{y}O_{z} perovskite lattice

| A | Ionic Radius | B | Ionic Radius | | Ionic Radius |
|---|---|---|---|---|---|
| Cation | (nm) | Cation | (nm) | Anion | (nm) |
| Ca⁺² | 0.134 | Mn⁺² | 0.067 | O⁻² | 0.14 |
| Sr⁺² | 0.144 | Mn⁺³ | 0.058 | S⁻² | 0.184 |
| Ba⁺² | 0.161 | Mn⁺⁴ | 0.053 | | |
| Na⁺ | 0.139 | Ti⁺⁴ | 0.0605 | F⁻¹ | 0.133 |
| K⁺ | 0.164 | V⁺³ | 0.064 | Cl⁻¹ | 0.181 |
| La⁺³ | 0.136 | V⁺⁴ | 0.058 | | |
| Bi⁺³ | 0.117 | Fe⁺² | 0.061 | | |
| | | Fe⁺³ | 0.055 | | |
| | | Ni⁺² | 0.069 | | |
| | | Ni⁺³ | 0.056 | | |
| | | Cu⁺¹ | 0.077 | | |
| | | Cu⁺² | 0.073 | | |
| | | Zn⁺² | 0.074 | | |
| | | Ce⁺³ | 0.101 | | |
| | | Ce⁺⁴ | 0.087 | | |
| | | Al⁺³ | 0.0535 | | |
| | | Sb⁺³ | 0.076 | | |

The perovskite crystal structure tolerates large distortions with respect to the ABO₃ cubic symmetry, allowing for changes in the ideal stoichiometry and for a wide variety of targeted metal substitutions/additions at both the A and B sites. The stability of the perovskite lattice can be determined by the Goldschmidt tolerance factor, t, given by

$$t = \frac{{r}_{A} + {r}_{O}}{\sqrt{2} ( {r}_{B} + {r}_{O} )}$$

where r_{A}, r_{B}, and r_{O} are the ionic radius of the A cation, B cation, and anion, respectively. When dopants are added at either the A or B sites and/or the charge on an ion changes, the average ionic radius is used in the calculation. t≈1 yields the idealized cubic perovskite lattice. When t is between ˜0.85 and 1.1, the lattice can deform slightly due to distortion, rotation, and tilting of the BO₆ octahedra. The perovskite lattice is still stable, however. Table 3 below shows representative compounds by substitution into the A, B, and anion sites, their Goldschmidt tolerance factor, and the theoretical average oxidation state of manganese (which may differ from the actual oxidation state due to the oxygen content). These compounds may be synthesized using solid-state or solution processes and may lead to greater or lesser spontaneous reactive oxygen species generation.

TABLE 3
Representative compounds by substitution into the A, B, and anion
sites, their Goldschmidt tolerance factor (t), and the theoretical
average oxidation state of manganese (which may differ from the
actual oxidation state due to the oxygen content)

| | | Theoretical Mn |
|---|---|---|
| Compound | t | Oxidation State |
| CaMnO₃ | 1.004 | +4 |

A site substitution (Ca)

| | | |
|---|---|---|
| SrMnO₃ | 1.041 | +4 |
| BaMnO₃ | 1.103 | +4 |
| Ca_{0.5}Na_{0.5}MnO₃ | 1.011 | +4.5 |
| Ca_{0.5}K_{0.5}MnO₃ | 1.059 | +4.5 |
| La_{0.65}Ca_{0.35}MnO₃ | 1.008 | +3.35 |
| Bi_{0.65}Ca_{0.35}MnO₃ | 0.964 | +3.35 |
| CeMnO₃ | 0.861 | +3 |

B site substitution (Mn)

| | | |
|---|---|---|
| CaMn_{0.8}Ti_{0.2}O₃ | 0.996 | +4 |
| CaMn_{0.8}V_{0.2}O₃ | 0.999 | +4 |
| CaMn_{0.8}Fe_{0.2}O₃ | 1.002 | +4.25 |
| CaMn_{0.8}Ni_{0.2}O₃ | 1.001 | +4.25 |
| CaMn_{0.8}Cu_{0.2}O₃ | 0.983 | +4.5 |
| CaMn_{0.8}Zn_{0.2}O₃ | 0.982 | +4.5 |
| CaMn_{0.8}Al_{0.2}O₃ | 1.003 | +4.25 |
| CaMn_{0.8}Bi_{0.2}O₃ | 0.981 | +4.25 |

Oxygen substitution

| | | |
|---|---|---|
| CaMnO_{2.4}S_{0.6} | 0.991 | +4 |
| CaMnO_{2.4}F_{0.6} | 1.005 | +3.4 |
| CaMnO_{2.4}Cl_{0.6} | 0.992 | +3.4 |

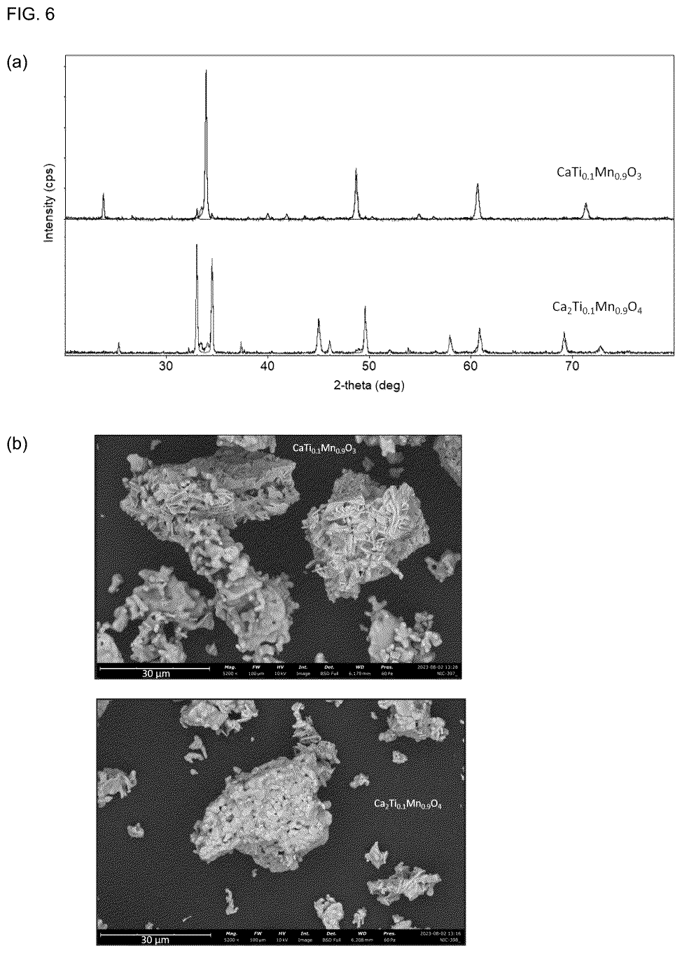

CaTi_{0.1}Mn_{0.9}O₃ and Ca₂Ti_{0.1}Mn_{0.9}O₄ were synthesized by solid state synthesis, where CaCO₃, MnCO₃ and TiO₂ (rutile) were mixed in a mortar and pestle and calcined at 1200° C. for 12 hours. CaMn_{0.8}Cu0.2O₃ was synthesized via the acetates route discussed in Example 3, where stoichiometric amounts of Ca(C₂H₃O₂)₂·H₂O, Mn(C₂H₃O₂)₂·4H₂O and Cu(C₂H₃O₂)₂·3H₂O were dissolved in distilled water, mixed and stirred for 20 minutes and the solution was allowed to evaporate in a drying oven at 80° C. overnight. The resulting mixed salt was heated to 400° C. for 2 hours and calcined at 1000° C. for 2 hours. FIG. 6 in (a) thereof shows the powder X-ray diffraction patterns of CaTi_{0.1}Mn_{0.9}O₃ (top) and Ca₂Ti0.1Mn_{0.9}O₄ (bottom) synthesized by the solid-state procedure, and scanning electron micrographs are shown in (b) of FIG. 6 for such CaTi_{0.1}Mn_{0.9}O₃ and Ca₂Ti_{0.1}Mn_{0.9}O₄ compounds.

### EXAMPLE 5

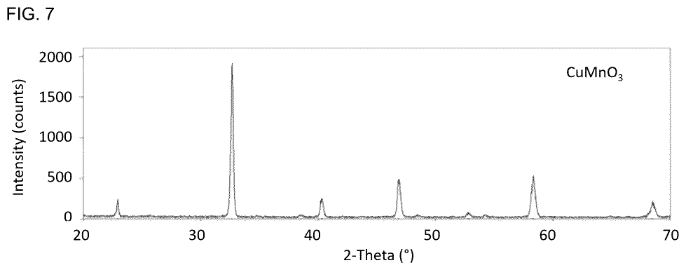

CuMnO₃ was synthesized by a solid-state method. Equimolar amounts of CuO and MnO₂ powders were ball-milled together and then calcined in air for 20 hours between 800 and 1100° C. The single phase, perovskite structure of the resulting material is confirmed by the powder X-ray diffraction pattern in FIG. 7. The sample generated 4.8 ppm of H₂O₂ at room temperature (˜25° C.) after 90 min. using the same test procedure outlined above.

### EXAMPLE 6

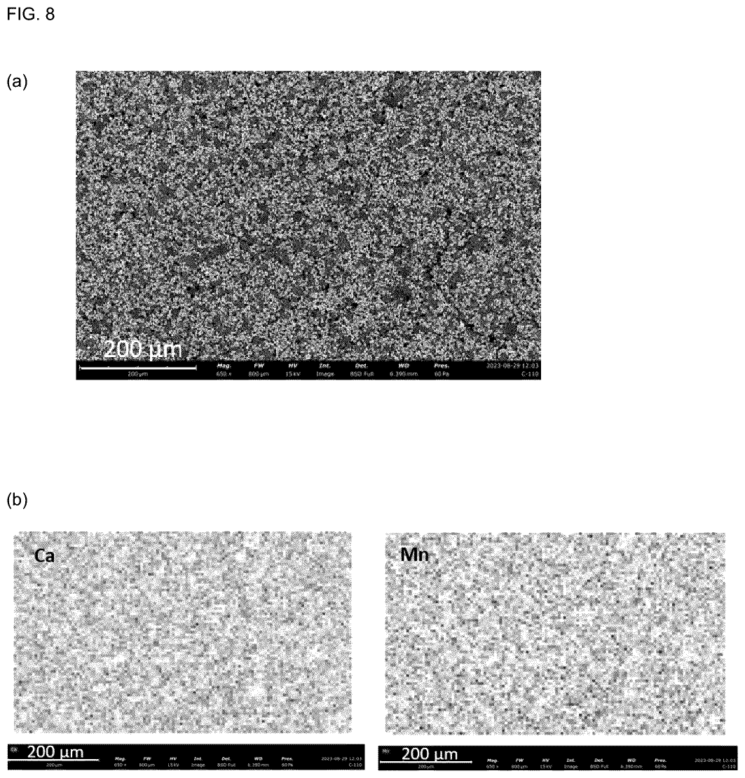

Mixed metal manganese oxide powders can be blended with paints and/or polymeric binders to form antimicrobial surface coatings since reactive oxygen species are known to inactivate pathogens. ROS can cause oxidative damage to proteins, nucleic acids, lipids, membranes, and organelles and can lead to rapid cell death even without the complete destruction of the biological species. As a representative example, FIG. 8 in (a) thereof shows a scanning electron micrograph of CaMnO_{3-δ} blended with a polyurethane/polyurea binder and coated at 10% by weight on an aluminum foil disc. FIG. 8 in (b) shows an energy dispersive X-ray analysis of both calcium (red) and manganese (purple) evidencing a uniform distribution of ceramic particles across the coating. Table 4 below is a summary of the inactivation of various pathogens using CaMnO_{3-δ}. The thickness of the dried coating was ˜40 microns, and exposure times varied from 5 to 10 minutes, with all experiments carried out at room temperature.

TABLE 4
Inactivation of selected pathogens by 10% weight
CaMnO_{3-d} in a polyurethane/polyurea binder (except C. albicans,
which was tested on CaMnO_{3-d} powder); all experiments
were carried out at room temperature

| | | |
|---|---|---|
| Pathogen | Time | Inactivation |

| | | | | |
|---|---|---|---|---|
| Bacteria | S. aureus | 10 | min | >99.9% |
| | E. coli | 10 | min | >99.9% |
| Fungus | C. albicans | 24 | h | 100% |
| Virus | Vaccinia | 5 | min | >99.9% |

### EXAMPLE 7

CaₓMn_{y}O_{z} and doped-CaₓMn_{y}O_{z} may be deposited on the surface of a particle using either physical or chemical means or combinations thereof. Physical deposition methods include, but are not limited to, sputtering, evaporation, plasma spray, thermal spray, laser deposition, etc. (see, for example, Handbook of Sputter Deposition Technology 2^{nd} Ed. (2012) and references cited therein). Chemical deposition methods include, but are not limited to, sol gel formation, chemical bath (liquid) deposition, chemical vapor deposition, plasma enhanced chemical vapor deposition, atomic layer deposition, electrochemical deposition (plating), mechano-chemical synthesis, etc. (see, for example, Chemical Solution Deposition of Functional Oxide Thin Films (2013) and references cited therein). Calcining after deposition may also be employed. The films may be continuous or discontinuous (porous) and may be uniform in structure or consist of small particles (and combinations thereof).

The composition, thickness, and morphology/structure of the film may be either homogenous or heterogeneous. A tie or pretreatment layer may be used to enhance the wetting of the particle surface, alter the morphology of the CaₓMn_{y}O_{z} or doped-CaₓMn_{y}O_{z} coating, and/or improve the adhesion of the coating. Average film thickness may vary between ˜1 nm and >1 micron.

The particulate core material in various embodiments may include materials such as silica, alumina, macroreticulate polymers, porous silicon, glass, or ceramic. In other embodiments, the particulate core material may be or comprise metal, metal alloys, carbon nanotubes, graphene, graphene oxide, or diatomaceous earth. Particle core materials may be selected for the provision of high surface area, to maximize presentation of the calcium manganese oxide and may lower costs by using cheap core materials. Active core materials such as metals/metal alloys, carbon nanotubes, graphene/graphene oxides may provide adjunctive or synergistic activity by affecting the electronic structure of the antimicrobial metal oxide.

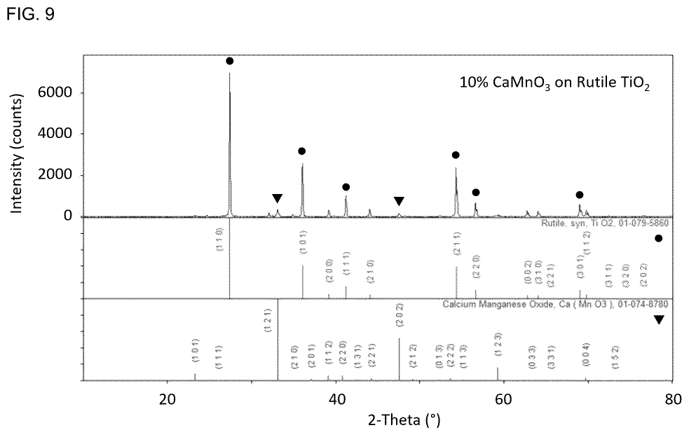

In one illustrative experiment, the coating on particles was deposited using a sol-gel process. Stoichiometric amounts of Ca(NO₃)₂·4H₂O and Mn(NO₃)₂·4H₂O were dissolved in distilled water, and citric acid was added as a ligand in 2:1 mole ratio to the metal ions. The solution was heated on an oil bath at 90° C. for 1 hour to evaporate the solvent and promote polymerization until a thick gel was formed. At this point, heating was removed and an amount of rutile TiO₂ powder was added to the gel in a 90% weight ratio and blended until a homogeneous mixture was obtained. The resulting mass was dried at 180° C. for 20 hours, ground in a mortar and pestle, and then calcined at 600° C. for 4 hours. The top panel of FIG. 9 shows a powder X-ray diffraction pattern of rutile TiO₂ particles coated with CaMnO₃. The main peaks are indexed to that of rutile TiO₂ (dots, middle panel) and CaMnO₃ (inverted triangles, lower panel) at a corresponding ration of approximately 9:1.

There have thus been described and illustrated certain embodiments of a metal oxide compound according to the disclosure. Although the metal oxide compounds and their preparation and applications have been described and illustrated in detail, it should be clearly understood that the disclosure is illustrative only and is not to be taken as limiting, the spirit and scope of the invention being limited only by the terms of the appended claims and their legal equivalents, as construed in the context of the specification herein. Thus, while the focus of the description herein has been on manganese compounds, this phenomenon can be observed in other transition metal oxides and mixed metal oxides as well, including, but not limited to, vanadium: +2, +3, +4, +5; chromium: +2, +3, +6; iron: +2, +3; cerium: +3, +4, cobalt: +2, +3; nickel: +2, +3, +4; and copper: +1, +2.

What is claimed is:

1. A material or blend of materials with an average stoichiometry CaₓMn_{y}O_{z} wherein x≥0.1, y≥0.1, and z≥1, which generates spontaneously reactive oxygen species.

2. The material or blend of materials of claim 1, blended with a material capable of a Fenton reaction.

3. The material or blend of materials of claim 1, having added thereto one or more alkali metal, alkaline earth, transition metal, lanthanide, and/or main group element(s).

4. The material or blend of materials of claim 1, wherein at least some of the oxygen is replaced by one or more halogen and/or chalcogenide atoms.

5. The material or blend of materials of claim 1, comprising a CaₓMn_{y}O_{z} perovskite lattice substituted with one or more A-site cation(s) selected from the group consisting of strontium, barium, sodium, potassium, lanthanum, cerium, and bismuth.

6. The material or blend of materials of claim 1, comprising a CaₓMn_{y}O_{z} perovskite lattice substituted with one or more B-site cation(s) selected from the group consisting of titanium, vanadium, iron, nickel, copper, zinc, cerium, aluminum, and antimony.

7. The material or blend of materials of claim 1, comprising a CaₓMn_{y}O_{z} perovskite lattice substituted with one or more anion(s) selected from the group consisting of sulfur, fluorine, and chlorine.

8. The material or blend of materials of claim 1, coated on the surface of an object.

9. The material or blend of materials of claim 1, coated on the surface of a particle.

10. The material or blend of materials of claim 1, mixed with a binder and coated on a surface.

11. A method of decomposing a chemical, inactivating a pathogen, or preventing biofouling at a biofouling-susceptible locus, comprising contacting the chemical, pathogen, or biofouling-susceptible locus with the material or blend of materials of claim 1.

12. A material or blend of materials with an average stoichiometry AeₓTm_{y}O_{z} wherein Ae is one or more alkaline earth elements, Tm is one or more transition metals or lanthanides with at least one having multiple valences, and x≥0.1, y≥0.1, and z≥1, which generates spontaneously reactive oxygen species.

13. The material or blend of materials of claim 12, blended with a material capable of a Fenton reaction.

14. The material or blend of materials of claim 12, wherein at least some of the oxygen is replaced by one or more halogen and/or chalcogenide atoms.

15. The material or blend of materials of claim 12, coated on the surface of an object.

16. The material or blend of materials of claim 12, coated on the surface of a particle.

17. The material or blend of materials of claim 12, mixed with a binder and coated on a surface.

18. A method of decomposing a chemical, inactivating a pathogen, or preventing biofouling at a biofouling-susceptible locus, comprising contacting the chemical, pathogen, or biofouling-susceptible locus with the material or blend of materials of claim 12.
